(19) United States
(12) Patent Application Publication
(10) Pub. No.: US 2022/0039975 A1
(43) Pub. Date: Feb. 10, 2022

(54) PROSTHESIS LINER

(71) Applicant: Ottobock SE & Co. KGAA, Duderstadt (DE)

(72) Inventors: Martin HILLMANN, Duderstadt (DE); Bernard GARUS, Einbeck (DE)

(73) Assignee: Ottobock SE & Co. KGAA, Duderstadt (DE)

(21) Appl. No.: 17/297,412

(22) PCT Filed: Nov. 27, 2019
(86) PCT No.: PCT/EP2019/082241

§ 371 Date: May 26, 2021

(30) Foreign Application Priority Data
Nov. 26, 2018 (DE) 102018129737.8

(51) Int. Cl. A61F 2/78

(52) U.S. Cl. CPC A61F 2/7812; A61F 2002/7818

(57) ABSTRACT

The invention relates to a prosthesis liner with a base body made of an elastic inner material which comprises an open proximal end, a closed distal end opposite the proximal end, and a longitudinal extension, which extends from the proximal end to the distal end, a bi-elastic textile layer arranged on or in the liner material, and a connection region that is arranged and configured to be connected to a prosthesis socket, wherein the textile layer has, in a section that extends proximally from the connection region, at least one tension element by means of which the elasticity of the textile layer is reduced in the longitudinal direction of the base body.

The invention relates to a prosthesis liner with a base body made of an elastic liner material, the base body having an open proximal end, a closed distal end opposite the proximal end and a longitudinal extension which extends from the proximal end to the distal end, a bi-elastic textile layer that is arranged on or in the liner material, and a connection region which is arranged and configured to be connected to a prosthesis socket.

Such prosthesis liners have been known from the prior art for many years. Prosthesis liners are used as an intermediate layer between a residual limb of the prosthesis wearer, also known as an amputation stump, and a prosthesis socket on which the actual artificial limb is arranged. Particularly in the case of leg prostheses, but also in the case of arm prostheses, it is very important in terms of the wearing comfort and the sense of security felt by the wearer to create a firm connection between the amputation stump and the prosthesis liner placed over it and the further prosthesis, in particular the prosthesis shaft, without causing pressure marks, painful wounds or chafing of one of the components against the wearer's skin.

Prosthesis liners are predominantly used in two different embodiments.

There are prosthesis liners that are pulled over the amputation stump, such that a volume between the prosthesis socket and the prosthesis liner that has been pulled over the amputation stump is hermetically sealed by inserting the prosthesis liner with the amputation stump into the prosthesis socket. A negative pressure can be created in this preferably hermetically sealed volume via a pump or by way of an outlet valve, said negative pressure holding the prosthesis socket in place on the amputation stump arranged in the liner. Here, a passive outlet valve is preferably arranged and designed in such a way that it is activated when walking with a prosthesis arranged on the prosthesis socket. Particularly during the swing phase of a gait cycle, large forces are generated that act on the prosthesis socket in the distal direction. Due to the negative pressure, they are transmitted relatively evenly onto the prosthesis liner and thus onto the amputation stump within. In this case, the connection region of the prosthesis liner is the region that abuts the volume in which the negative pressure is created. The connection region usually extends in a proximal direction from the distal end of the prosthesis. In some embodiments of the prosthesis liner, at least one, but possibly multiple, sealing lips are arranged on an outer side of the base body. These lips limit the volume in which the negative pressure is created and thus also the connection region in a proximal direction.

An alternative embodiment of the liner features a cap or cup, in particular at its distal end, on which a fastening element is arranged that may be designed, for example, in the form of a distally protruding pin. This fastening element is used to engage with a correspondingly designed fastening element inside the prosthesis socket, thereby establishing as secure a connection as possible between the prosthesis liner and the prosthesis socket. With this type of connection between prosthesis socket and prosthesis liner, arising tensile forces that are to be transmitted from the prosthesis socket to the prosthesis liner are almost entirely transmitted by way of the fastening elements. Consequently, the tensile forces that occur act almost exclusively on the distal region of the liner, meaning that on the one hand this region is also subjected to high mechanical stress and on the other hand the milking effect on the amputation stump, which is uncomfortable for the wearer, occurs particularly in this region due to the strongly changing load. In this case, the connection region is limited to the distal cup or cap.

This effect is intensified since the liner material that the base body of the prosthesis liner is made of is elastic. It is, for example, a silicone, a thermoplastic elastomer (TPE) or a polyurethane that is designed to be elastic both in the longitudinal extension of the base body and in the circumferential direction of the base body. This elasticity is necessary to be able to produce suitable standard liners for amputation stumps of various shapes and sizes. It is also necessary to expand the liner when pulling it onto or mounting it on the amputation stump in order to be able to guarantee the required holding force.

EP 2 538 892 describes a prosthesis liner whose textile layer is composed of two different textiles. A first textile, which is made of a bi-elastic material, is arranged in the region of a joint, such as a knee, which is covered and enclosed by the prosthesis liner in the mounted state. In this context, bi-elastic means that the material is designed to be elastic in both the longitudinal extension as well as the circumferential direction of the base body. In all other regions, the textile layer is made of a different textile that is designed to be mono-elastic. Elasticity in the longitudinal extension of the base body is severely limited, but elasticity is present in the circumferential direction.

The disadvantage is that, due to the plurality of textile elements to be used which have to be sewn together before being connected to the base body, the production of such a prosthesis liner is complex and therefore costly. In addition, the necessary seams that connect the individual textile elements may cause pressure marks or a reduced degree of comfort when wearing the liner.

US 2005/0149202 A1 discloses a liner with a stocking-like base body made of a woven fabric. A coating is applied to the outer side of this base body in the region of the distal cap, said coating exhibiting a lower elasticity than the woven fabric. In another configuration shown, multiple strips made of a woven fabric or textile are applied instead of the coating, wherein said fabric or textile exhibits a reduced elasticity compared to the woven fabric of the base body. In this case, too, the fact that multiple textile elements are to be connected to one another results in seams which feature the disadvantages specified above.

U.S. Pat. No. 6,231,617 B1 describes a liner made of an elastic plastic, such as a urethane or silicone. Multiple arms made of a material that is inelastic in the longitudinal direction are embedded in this material, wherein such arms may be made, for example, of a metal, a plastic, cotton or carbon fibers. U.S. Pat. No. 4,923,474 B1 contains a similar principle. In this case, too, inelastic elements are embedded in a base body made of an elastic material, such as a rubber material, said inelastic elements reducing the elasticity of the liner in the longitudinal direction.

The invention aims to improve a prosthesis liner according to the preamble of claim 1 in such a way that the disadvantages named are avoided or at least mitigated, and the liner is simple and inexpensive to produce.

The invention solves the problem by way of a prosthesis liner according to the preamble of claim 1, which is characterized in that the textile layer comprises at least one tension element in a section that extends from the connection region in the proximal direction, said tension element reducing the elasticity of the textile layer in the textile direction of the base body.

Preferably, the section in which the textile layer comprises the at least one tension element extends from the distal end of the textile layer. In this case, the connection region is preferably formed by a distal cap with at least one fastening element for fastening the prosthesis liner to a prosthesis socket.

Alternatively or additionally, the prosthesis liner features at least one sealing lip which limits the connection region in the proximal direction. The sealing lip is preferably arranged and designed in such a way that, when the liner is in the mounted state, it comes into contact with an inner wall of a prosthesis socket when the amputation stump over which the prosthesis liner has been pulled is inserted into the prosthesis socket. The contact between the inner wall of the prosthesis socket and the sealing lip then preferably ensures that a volume extending distally from the sealing lip is hermetically sealed, so that the required negative pressure can be created therein.

In a preferred embodiment of the invention, the at least one tension element extends into the connection region. It is then the same tension element that extends into the connection region and proximally from the connection region. Alternatively or additionally, the textile layer also comprises at least one tension element in the connection region, by way of which the elasticity of the textile layer is reduced in the longitudinal direction of the base body.

The textile layer preferably features a plurality of tension elements, which are particularly preferably distributed equidistantly across a circumference of the textile layer. In an alternative embodiment, the tension elements are arranged exclusively in the neutral fiber of the prosthesis liner. Conventionally, prosthesis liners are designed in such a way that they enclose a joint of the wearer of the prosthesis liner, such as a knee joint. If the joint is flexed, one part of the enclosing prosthesis liner is elongated and another part is compressed. In the case of a knee joint, when the joint is bent (flexion), the front part of the prosthesis liner (ventral) is elongated and the rear part (dorsal) is compressed. There are also areas that are neither elongated nor compressed. In this example, these are located medially and laterally to the joint and referred to as “neutral fibers”.

This embodiment is especially, but not exclusively, advantageous for prosthesis liners that feature fastening elements or other connection devices in their distal region for connecting the prosthesis liner in this region to a prosthesis socket or another prosthesis element. Arising tensile forces, which may occur, for example, in the swing phase of a gait cycle, are introduced into the distal region of the prosthesis liner and the textile layer via the connecting elements and fastening elements. They act in the longitudinal extension of the prosthesis liner. The elasticity of the liner material is greatly reduced or completely eliminated in the section extending from the distal end of the textile layer by the use of the at least one tension element. In the area in which tensile forces are introduced into the prosthesis liner and thus the textile layer, for example during the swing phase of a step, the textile layer is inelastic in this direction, such that the tensile forces are transmitted via the at least one tension element and distributed in a larger area, namely in particular a part of the section in which the at least one tension element is located. This results in a more even distribution of the occurring forces and therefore a reduction in the pressure acting on the amputation stump. This pressure is also distributed more evenly, thereby increasing the degree of comfort experienced when wearing the liner.

Advantageously, the tension elements comprise fibers and/or threads made of an inelastic material. For example, an inelastic yarn can be used, which is inserted into the textile and already incorporated during the production of the textile. The threads preferably run parallel to the longitudinal extension, i.e. they begin at the distal end of the textile layer and extend towards the proximal end of the base body. The textile layer is preferably arranged on an outer side of the base body. Advantageously, the textile layer covers the entire outer side of the base body, so that the open proximal end of the base body is also surrounded by a proximal end of the textile layer that is also open. Advantageously, the tension elements, which begin at the distal end of the textile layer, extend towards this proximal end. By using an inelastic material as a tension element, the elasticity of the textile layer can be almost or even completely eliminated in this direction. The textile layer is then inelastic in the longitudinal extension. Given that the textile layer is connected to the base body made of the elastic liner material over as full an area as possible, also the base body cannot be stretched in the direction of its longitudinal extension in this area either, so that the elasticity of the base body along its longitudinal extension has been greatly reduced or even completely eliminated in this section, which extends from the distal end.

In a preferred embodiment, the tension elements are fastened to the textile layer at their respective proximal ends. In this way, tensile forces acting on the tension elements can be transferred and transmitted to the textile layer. The proximal end of the tension elements is preferably situated at a distance from the proximal end of the base body. This means that the tension elements do not extend across the entire longitudinal extension of the textile layer and thus also not across the entire longitudinal extension of the base body. Consequently, the prosthesis liner comprises the previously specified section, which extends from the distal end and in which an elasticity of the prosthesis liner along the direction of the longitudinal extension has been greatly reduced or even completely eliminated, and a remaining section in which the tension elements do not extend, in that the original elasticity of the bi-elastic textile layer and elastic liner material is still present. Particularly in this remaining section of the prosthesis liner, there is still an elasticity present along the longitudinal extension.

Preferably, the tension elements feature a loop at their proximal end, said loop being guided around at least one element of the textile layer. If the textile is, for example, a woven fabric, the loop is preferably guided around at least one weft thread and/or at least one warp thread.

Tension elements are preferably made of a thread or yarn that is preferably inelastic. This thread has two ends and is preferably double-laid to form the tension element, so that its two ends form one end of the tension element and a loop forms the opposite end. The end formed by the loop forms the proximal end of the tension element and is preferably guided around a part of the textile layer, such that a tensile force acting on the tension element is transmitted via the loop to the textile layer. The two ends of the thread that form the distal end of the tension element are preferably guided out of the textile layer at the distal end of the prosthesis liner. If the prosthesis liner is to be adapted to individual conditions of an amputation stump, a tension and thus a deformation can be exerted on the textile layer and thus on the base body attached to it or surrounding it by applying a tensile force to the distal ends of the tension elements. This renders it possible to customize the prosthesis liner to at least a limited degree.

Preferably, the tension elements are guided at least in sections within the textile layer. On the one hand, this prevents the tension elements from being located exclusively on the outer side of the textile layer between their distal end and their proximal end, and thus, where applicable, on the outer layer of the prosthetic liner. In this case, there would be a risk, for example, of the user of the prosthesis liner hooking into the loops formed in this way when mounting the liner and causing damage due to the forces that are then acting. Guiding the tension elements at least in sections within the textile layer can enable further connections between the respective tension element and the textile layer, so that tensile forces acting on the tension element can not only be transmitted to the textile layer at its proximal end, but also potentially at intermediate points situated between the proximal end and the distal end of the tension elements.

It is especially preferable if the prosthesis liner has a distal cap with at least one fastening element for fastening the prosthesis liner to a prosthesis socket, wherein the distal ends of the tension elements are bonded, preferably glued or moulded, to the distal cap. Before the distal ends of the tension elements are connected to the distal cap, tension is preferably exerted on the distal ends of the tension elements in the manner previously described in order to carry out a forming and adjustment of the section of the prosthesis liner to fit the individual conditions of the user of the prosthesis liner. Following these adjustments, the distal ends of the tension elements can be bonded, for example glued or moulded, to the distal cap, such that they cannot be separated and a subsequent change, in particular an accidental or incorrect one, remains impossible. This also guarantees an especially effective force transmission between the distal cap on which the tensile forces act, for example in the swing phase of a step, and the tension elements.

The cap is preferably integrated into the prosthesis liner; it is preferably cast or injected into the elastic liner material.

The prosthesis liner is preferably designed to enclose a joint, especially a knee or an elbow, of an amputation stump when in the mounted state, with the section terminating distally of the joint. The section of the textile layer in which the tension elements are located is thus preferably arranged completely distally to the respective joint. This ensures that elasticity is present along the longitudinal extension of the liner in the region of the respective joint, especially the knee, so as to be able to apply the necessary extension that inevitably occurs when the respective joint is bent.

Particularly, but not exclusively, in the event that the section with the tension elements is not located exclusively distally to a joint of the wearer, it is advantageous for the tension elements to be situated at least in the area of the joint that is medial and/or lateral to the joint, particularly in the neutral fibers. In a preferred embodiment, the tension elements are arranged distally to the joint and possibly also proximally to the joint and equidistantly across the circumference, and medially and/or laterally to the joint in the region of the joint.

The invention also solves the problem by way of a system composed of a prosthesis liner according to one of the embodiments described herein and a prosthesis socket, the prosthesis socket being designed in such a way that it can be connected to the connection region of the prosthesis liner.

In the following, some examples of embodiments of the present invention will be explained in more detail by way of the attached figures:

They show:

FIGS. 1 to 3—various examples of embodiments of a prosthesis liner, each in a frontal view and a side view,

FIG. 4—the schematic representation of a knitted fabric that can be used for such a liner, and

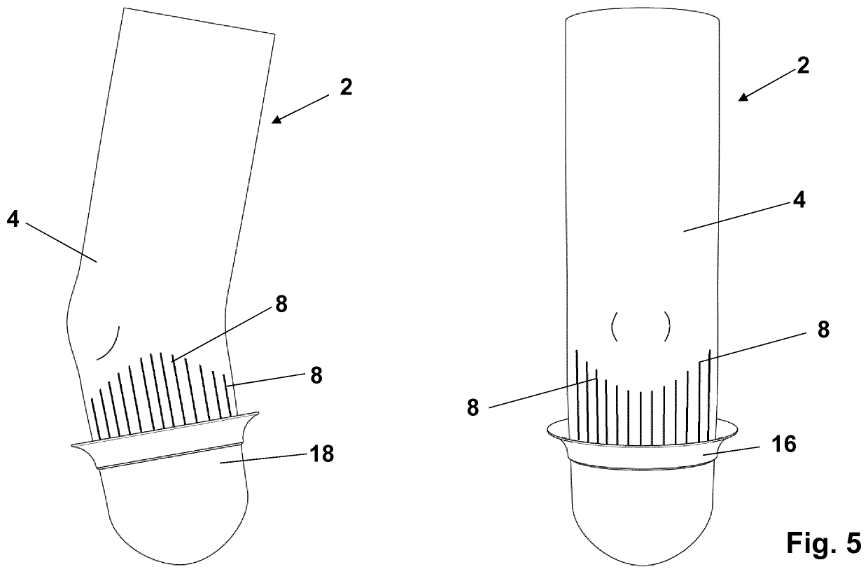

FIG. 5—a further example of an embodiment of a prosthesis liner in a frontal view and a side view.

FIG. 1 shows a prosthesis liner 2 with a base body 4 with a textile layer, not explicitly shown here, located in its distal area. The prosthesis liner 2 comprises a connecting cap 6 on which fastening elements can be arranged in order to arrange the prosthesis liner 2 in a prosthesis socket, not depicted.

Multiple tension elements 8, represented as dashed lines, are arranged in the bielastic textile layer arranged on the base body 4 of the liner 2. They are filler threads which are stitched into the textile of the textile layer. They are preferably made of an inelastic material or a material with a lower elasticity than the textile layer.

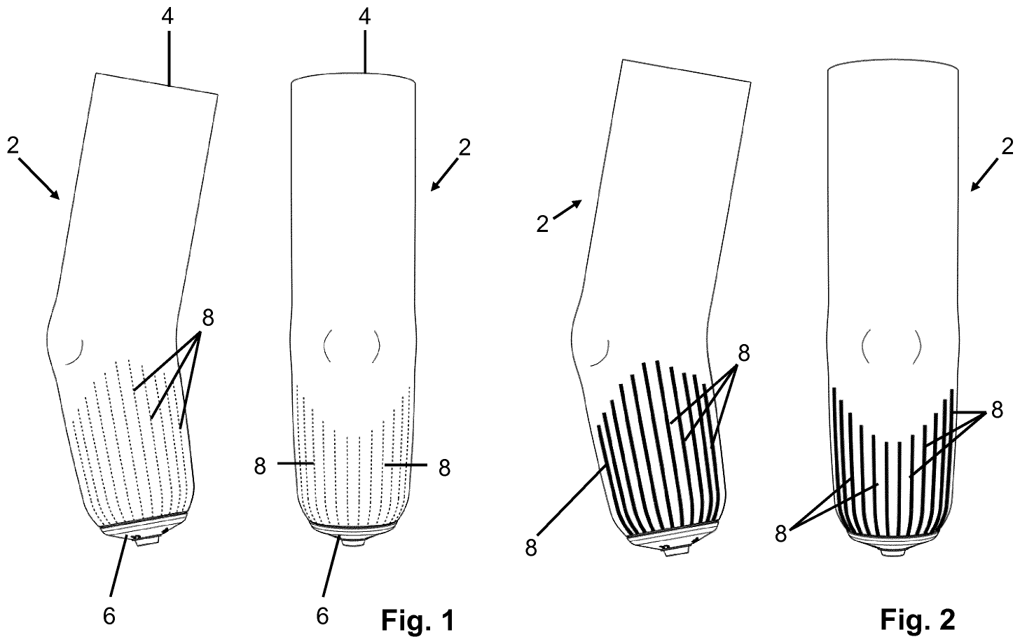

FIG. 2 depicts a different embodiment of the prosthesis liner 2, again with a textile layer, not depicted, arranged in the distal area. Instead of the stitched filler threads from FIG. 1, in FIG. 2 tension elements 8 have been inserted into the textile layer that have a thermoplastic coating or melt fibers, or are made of a thermoplastic material themselves. Once the tension elements 8 in the form of filler threads have been inserted into the textile material of the textile layer, they can be melted or at least softened to the extent that they bond with the textile layer. The particularly special feature is that this bond occurs not on the outer side of the textile layer, but within the textile layer, as the filler threads have first been integrated in the textile layer and incorporated into it.

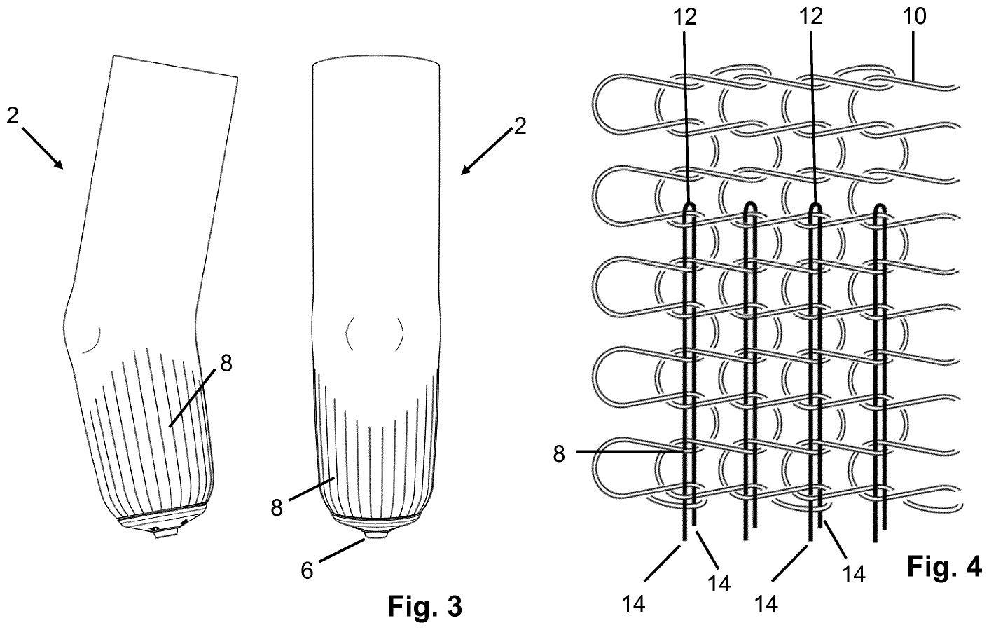

FIG. 3 depicts a further embodiment of the prosthesis liner 2 in which the tension elements 8 are introduced into the textile of the textile layer, not depicted, in the form of filler threads.

FIG. 4 schematically depicts how the tension elements 8 are positioned in the knitted fabric. The knitted fabric 10 can be seen that forms part of the elastic textile layer arranged on or in the liner material of the prosthesis liner 2. The loops of the knitted fabric 10 are formed of a yarn made of a first material or a material mix. In both directions, i.e. upwards and downwards as well as to the right and left in FIG. 4, the knitted fabric 10 exhibits an elasticity which makes it a bi-elastic textile. The tension elements 8 in the form of filler threads are introduced into the knitted fabric from the distal end and extend in the distal direction until they form a guide loop and are redirected back in the distal direction parallel to the previous path. The two ends can be connected, for example glued, to each other at the distal end or connected to the distal connecting cap 6.

FIG. 5 shows the prosthesis liner 2 in a front view and a side view, the base body 4 of which comprises a plurality of tension elements 8, which, however, do not extend to the distal end of the prosthesis liner 2. The prosthesis liner 2 depicted features a sealing lip 16 that comes into contact with an inner wall of a prosthesis socket when the prosthesis liner 2 is inserted into the prosthesis socket when the liner is in the mounted state. As a result, a volume is hermetically sealed distally of the sealing lip 16, i.e. below the sealing lip 16 in the representations shown, in which a negative pressure can be generated by which the prosthesis socket is held on the prosthesis liner 2. The connection region 18 extends in this area distal to the sealing lip 16.

### REFERENCE LIST

2 prosthesis liner
4 base body
6 connecting cap
8 tension element
10 knitted fabric
12 guide loop
14 end
16 sealing lip
18 connection region

1. A prosthesis liner with
a base body made of an elastic liner material which comprises an open proximal end, a closed distal end opposite the proximal end, and a longitudinal extension, which extends from the proximal end to the distal end;
a bi-elastic textile layer arranged on or in the liner material, and
a connection region that is arranged and configured to be connected to a prosthesis socket;
wherein the textile layer has, in a section that extends proximally from the connection region, at least one tension element by means of which the elasticity of the textile layer in the longitudinal direction of the base body is reduced.

2. The prosthesis liner according to claim 1, wherein the section preferably extends from the distal end, wherein the connection region is preferably formed by a distal cap with at least one fastening element for fastening the prosthesis liner to a prosthesis socket.

3. The prosthesis liner according to claim 1, wherein the connection region is limited proximally by at least one sealing lip.

4. The prosthesis liner according to claim 1, wherein the at least one tension element extends into the connection region and/or wherein the textile layer also features at least one tension element in the connection region as well, by way of which the elasticity of the textile layer is reduced in the longitudinal direction of the base body.

5. The prosthesis liner according to claim 1, wherein the textile layer features a plurality of tension elements which are preferably distributed equidistantly across a circumference of the textile layer.

6. The prosthesis liner according to claim 1, wherein the tension element comprises fibers and/or threads made of an inelastic material.

7. The prosthesis liner according to claim 5, wherein the tension elements are fastened at their proximal end, which is preferably located at a distance from the proximal end of the base body, to the textile layer in such a way that tension forces acting on the tension elements in the longitudinal direction are transmitted to the textile layer.

8. The prosthesis liner according to claim 7, wherein the tension elements form a loop at their proximal end, which is guided around at least one element of the textile layer.

9. The prosthesis liner according to claim 1, wherein the tension elements extend at least in sections within the textile layer.

10. The prosthesis liner according to claim 1, wherein the prosthesis liner has a distal cap with at least one fastening element for fastening the prosthesis liner to a prosthesis socket, wherein the distal ends of the tension elements are bonded, preferably glued or moulded, to the distal cap.

11. The prosthesis liner according to claim 10, wherein the distal cap is integrated into the prosthesis liner, such as cast or injected into the elastic liner material.

12. The prosthesis liner according to claim 1, wherein the prosthesis liner is designed to enclose a joint, especially a knee or an elbow, of an amputation stump when in the mounted state, with the section terminating distally of the joint.

13. A system composed of a prosthesis liner according to claim 1, wherein a prosthesis socket is designed in such a way that it can be connected to the connection region of the prosthesis liner.

14. A prosthesis liner comprising:
a base body made of an elastic liner material, the base body comprising:
an open proximal end,
a closed distal end opposite the proximal end, and
a longitudinal extension which extends from the proximal end to the distal end;
a bi-elastic textile layer arranged on or in the liner material; and
a connection region configured to be connected to a prosthesis socket;
wherein the textile layer has at least one tension element in a section that extends proximally from the connection region, the tension element configured to reduce the elasticity of the textile layer in the longitudinal direction of the base body; and
wherein the textile layer also has at least one tension element in the connection region, the tension element configured to reduce the elasticity of the textile layer in the longitudinal direction of the base body.

15. The prosthesis liner of claim 14, wherein the textile layer features a plurality of tension elements distributed across a circumference of the textile layer.

16. The prosthesis liner of claim 15, wherein the plurality of tension elements are distributed equidistantly across the circumference of the textile layer.

17. The prosthesis liner of claim 15, wherein the tension elements comprise fibers and/or threads made of an inelastic material.

18. The prosthesis liner of claim 15, wherein the tension elements are fastened at their proximal end, which is preferably located at a distance from the proximal end of the base body, to the textile layer in such a way that tension forces acting on the tension elements in the longitudinal direction are transmitted to the textile layer.

19. The prosthesis liner of claim 15, wherein the prosthesis liner has a distal cap with at least one fastening element for fastening the prosthesis liner to a prosthesis socket, wherein the distal ends of the tension elements are bonded, preferably glued or moulded, to the distal cap.

20. A prosthesis liner comprising:
a base body made of an elastic liner material, the base body comprising:
an open proximal end,
a closed distal end opposite the proximal end, and
a longitudinal extension which extends from the proximal end to the distal end;
a bi-elastic textile layer arranged on or in the liner material;
a connection region configured to be connected to a prosthesis socket; and
a distal cap with at least one fastening element for fastening the prosthesis liner to a prosthesis socket;
wherein the textile layer has a plurality of tension elements distributed across a circumference of the textile layer and extending proximally from the connection region, the tension elements comprising fibers and/or threads of an inelastic material, the tension elements being configured to reduce the elasticity of the textile layer in the longitudinal direction of the base body.
